(19) United States
(12) Patent Application Publication
(10) Pub. No.: US 2009/0018360 A1
(43) Pub. Date: Jan. 15, 2009

(54) PROCESS FOR PREPARATION OF ALKYL AND ALCOXYALKYL-ALPHA-CYANOACRYLATES BY DEPOLYMERISATION OF POLY(ALKYL-ALPHA CYANOACRYLATES) OR POLY(ALCOXYALKYL-ALPHA-CYANOACRYLATES) AND ITS USAGE AS TECHNICAL AND/OR MEDICAL ADHESIVE

(76) Inventors: Zlatan Zlatev Denchev, Guimaraes (PT); Milena Ivanova Tomanova, Guimaraes (PT); Antonio Magalhaes Da Cunha, Guimaraes (PT)

Correspondence Address: SUGHRUE MION, PLLC, 2100 PENNSYLVANIA AVENUE, N.W., SUITE 800, WASHINGTON, DC, 20037, US

(21) Appl. No.: 11/913,754

(22) PCT Filed: May 5, 2006
(86) PCT No.: PCT/IB06/51426

§ 371 Date: Aug. 22, 2008

(30) Foreign Application Priority Data
May 6, 2005 (PT) 103272

(51) Int. Cl. C07C 253/32

(52) U.S. Cl. 558/441

(57) ABSTRACT

This invention is related to the preparation of alkyl or alkoxyalkyl-α-cyanoacrylates in monomeric form by depolymerisation of the corresponding poly(alkyl-α-cyanoacrylates) or poly(alkoxyalkyl-α-cyanoacrylates) (PCA). The PCA's are obtained preferably by base-catalyzed condensation of a cyanoacetate with formaldehyde (or a polymer of the latter). According to the invention, the poly(alkyl-α-cyanoacrylate) or poly(alkoxyalkyl-α-cyanoacrylate), the condensation product, is mixed with a depolymerisation system comprising phosphorus pentoxide P₂O₅, hydroquinone, ortho-phosphoric acid and para-toluenesulfonic acid. This process is realized in a batch reactor fitted with a condenser, heated at a temperature in the range of 100-300° C., under a vacuum of 0.5-50 Torr (7.10⁻⁵-7.10⁻³ MPa) and gives rise to monomeric alkyl-α-cyanoacrylates or monomeric alkoxyalkyl-α-cyanoacrylates, being stable in both gaseous and liquid phase. After an additional purification by vacuum distillation, these cyanoacrylates can be used as fast setting technical or medical adhesives.

### FIELD OF THE INVENTION

This invention relates to the preparation of α-cyanoacrylates in monomeric form and is particularly concerned with the depolymerisation of poly(alkyl-α-cyanoacrylates) or poly(alkoxyalkyl-α-cyanoacrylates) (PCA) to produce monomers applicable in technical or medical adhesives. More specifically, the invention discloses an improved method for controlled depolymerisation of polymeric cyanoacrylates in the presence of a depolymerisation system containing specified amounts of phosphorous pentoxide (P₂O₅), hydroquinone, ortho-phosphoric acid and para-toluenesulfonic acid. The depolymerisation method disclosed allows the attainment of high-yield and high-purity alkyl or alkoxyalkyl-α-cyanoacrylates in reactors of simple design, with a minimum quantity of liquid waste and without the use of hazardous gases.

### BACKGROUND OF THE INVENTION

Cyanoacrylate monomers, when applied as a thin layer between two surfaces made out of similar or different materials, e.g. metals, polymers (with the exception of polyolefins), wood, stone, living tissue, etc. are able to bond to them rapidly and without the use of heat or catalysts. Conventionally, the esters of the α-cyanoacrylates are prepared using a two-stage reaction. In the first stage, the corresponding cyanoacetate is made to react with formaldehyde (as gas, aqueous solution or in polymeric form) in a basic environment to form poly(alkyl-α-cyanoacrylates). In the second stage, the polymer is cracked (i.e., depolymerised), purified and stabilized to obtain the cyanoacrylate adhesive.

The above reaction scheme was proposed for the first time by Ardis in U.S. Pat. No. 2,467,927, which discloses the condensation of formalin (ca. 40 wt. % of formaldehyde in water) with alkyl cyanoacetate using basic catalysts. The polymer thus formed is depolymerised in the presence of catalytic amounts of P₂O₅, employing a gaseous inhibitor of the back polymerization-sulphur dioxide SO₂ or nitric oxide NO. Later U.S. Pat. No. 2,721,858 by Joyner and Hawkins discloses the substitution of formalin, in the stage of condensation, with a formaldehyde polymer and the combined use of phosphorus pentoxide and hydroquinone in the stage of depolymerisation. Stable monomers were only obtained when SO₂-gas was passed through the reactor during the depolymerisation stage.

U.S. Pat. No. 2,756,251 by Joyner and Shearer describes a similar depolymerisation system comprising phosphorous pentoxide and hydroquinone (polymerization inhibitors for the liquid cyanoacrylate). The SO₂-gas is equally used to inhibit the spontaneous polymerization of the cyanoacrylate vapours. The use of high-boiling tertiary phosphoric acid esters as reaction media for depolymerisation, e.g., tricresyl phosphate, was here proposed for the first time. These phosphate esters should have boiling temperatures about 60-80° C. higher than the depolymerisation temperature. The amount of tricresyl phosphate required in the process disclosed in this patent is about 0.9 times that of the polymeric raw material, thus creating significant amounts of liquid waste. The reaction is conducted as a batch process.

An analogous method for making alkyl-α-cyanoacrylates was proposed by Robert Rabinowitz in U.S. Pat. No. 3,444,233 disclosing continuous depolymerisation of poly(alkyl-α-cyanoacrylate) esters. The polymer is first admixed with an inert liquid of high boiling point, such as tricresyl phosphate adding one or more polymerization inhibitors, such as phosphorus pentoxide and hydroquinone. This mixture is then depolymerised by heating a thin layer of this mixture in vacuum to produce high-purity monomeric α-cyanoacrylate vapours. The continuous process disclosed in this patent requires the use of large amounts of tricresyl phosphate (1.45 times the amount of the polycyanoacrylic acid esters), thus resulting in serious liquid pollution and disposal problems. Again, to get stable monomeric alkyl cyanoacrylates useful as instant adhesives, the depolymerisation has to be carried out in a stream of the hazardous and corrosive SO₂-gas.

U.S. Pat. No. 5,436,363 by Wang et al. discloses a continuous process for making cyanoacrylates, which utilizes a thin-layer evaporator and a two-condenser heat transfer system. A poly(alkyl-α-cyanoacrylate) feed containing catalytic amounts of P₂O₅ and hydroquinone without a high-boiling medium is introduced into a thin-film evaporator so as to carry out the depolymerisation reaction. The monomeric vapours produced by this process, where no high-boiling medium is used, do not require stabilization by SO₂-gas. They are then subjected to a two-stage heat transfer process for condensation and purification. The first stage involves a high temperature condenser operating at ca. 150° C. that collects a fraction containing primarily ‘dimers’ of cyanoacrylate (i.e., dicyanoglytarates). Subsequently, the gaseous phase enters a low-temperature condenser, operating at −8° C., where the cyanoacrylate monomer is collected. The advantages of this scheme employing thin layer evaporator and an intermediate condenser are: (i) the elimination of the need of using a high-boiling heat transfer medium; (ii) the elimination of the use of hazardous gases (SO₂ or NO); (iii) the attainment of a high-yield and high-purity final product of cyanoacrylate monomers. The major disadvantages of the proposed process are related to: (i) a quite complex reactor design; (ii) the need for a continuous, large-scale production, which is not always appropriate when smaller volumes of monomeric cyanoacrylates are needed, as in the case of medical adhesives.

Other reaction schemes, not including condensation or depolymerisation processes, are also worth mentioning:
1. the protection of the cyanoacrylate monomer in the condensation stage, by formation of its anthracene Diels-Alder adduct, with subsequent cyanoacrylate liberation, involving the reaction of the adduct with maleic anhydride, avoiding the stage of depolymerisation and originating stable alkyl-cyanoacrylates, useful as industrial adhesives, as exemplified by U.S. Pat. No. 3,463,804 by Ray and Doran and U.S. Pat. No. 4,012,402 (Buck);
2. the transesterification of the cyanoacrylate monomers with alcohols, as described in USSR authorship certification No 726086 by Y. B. Vojtekunas et al.;
3. the direct esterification of cyanoacrylic acid with alcohols is reported in German patent 3,415,181 by Schülter and Marten;
4. the thermal decomposition of alkyl 2-cyano-3-alkoxypropionates and the 3-acyloxy analogues are described in U.S. Pat. No. 2,467,926 by Ardis;
5. the pyrolysis of the cyanohydrin acetates of pyruvic acid esters is described in U.S. Pat. No. 2,391,251 by Long.

The major disadvantages of all these processes are their high complexity, requiring the isolation and the purification of many intermediates and their very long duration (typically 10-16 h per batch), which renders them unsuitable for industrial applications. Especially inappropriate for the synthesis of medical adhesives is the use of Diels-Alder protection that requires the implementation of readily sublimating carcinogenic compounds with condensed benzene rings that can migrate into the final adhesive and enter in contact with living tissue.

### SUMMARY OF THE INVENTION

The present invention discloses a versatile process for the production of alkyl and alkoxyalkyl-α-cyanoacrylates, applicable on technical and medical adhesives, by introducing a new stabilization system at the stage of depolymerisation. The general formula of these compounds is the following:

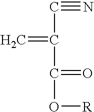

General formula of the alkyl or alkoxyalkyl-α-cyanoacrylates R=alkyl C₁-C₁₆ or alkoxyalkyl residue.

The new process proposed is simple in terms of reactor design, does not involve the generation of much liquid waste and excludes the use of hazardous inhibition gases.

### DETAILED DESCRIPTION OF THE INVENTION

The new process disclosed in the present patent makes use of the aforementioned condensation-depolymerisation scheme, employing a new stabilization system in the stage of depolymerisation, which comprises phosphorus pentoxide, hydroquinone, ortho-phosphoric acid and para-toluenesulfonic acid, in specific concentrations, as indicated hereafter. The co-inventors have identified the strong stabilization effect of the said four-component system, eliminating significantly the undesired back polymerization of the monomeric cyanoacrylates (in both liquid and gaseous state) in the depolymerisation stage. Thus, the polymer phase remains less viscous and better depolymerisable during the entire depolymerisation, without the necessity of use of high-boiling solvents. Furthermore, the cyanoacrylate vapours do not polymerize spontaneously, avoiding the use of gaseous polymerization inhibitors, such as SO₂. It is an advantage of the process proposed that it allows the use of a one-condenser batch reactor of a versatile and simple design, useful for the small- and medium-scale production of more sophisticated cyanoacrylate monomers applied as medical adhesives. Another advantage of the process proposed is the fact that it does not require the use of non-recyclable, high-boiling point solvents (such as tricresyl phosphate) and/or dangerous, corrosive gases (SO₂) which required special safety and purification procedures.

According to the process of the present invention, the poly(alkyl-a-cyanoacrylate) or poly(alkoxyalkyl-a-cyanoacrylate) condensation product is mixed with a depolymerisation system comprising phosphorous pentoxide (P₂O₅), hydroquinone, orthophosphoric acid (crystalline or liquid) and para-toluenesulfonic acid in a one condenser batch reactor, heated within the range of 100-300° C., preferably 150-250° C. and more preferably 180-220° C., and operating under a vacuum within the limits of 50-0.5 Torr (7×10⁻³−7×10⁻⁵ MPa), preferably 20-1 Torr (3×10⁻³−1.3×10⁻⁴ MPa) and more preferably 10⁻⁵ Torr (1.3×10⁻³−7×10⁻⁴ MPa), both depending on the structure of the monomer to be prepared.

Also depending on the structure of the monomer to be prepared, the relation between the phosphorous pentoxide and the hydroquinone is in the range, wt. parts, from 1:10 to 10:1, preferably between 1:1 and 1:5, more preferably between 1:1 and 1:3, and the relation between the ortho-phosphoric acid and para-toluenesulfonic acid is in the range from, wt. parts, 20:1 and 1:20, preferably between 15:1 and 10:1.

The present invention will now be described more specifically with reference to some typical examples. It is to be noted that the following examples are presented herein for the purpose of illustration and description and are not intended to be exhaustive or to limit the invention to the precise form of the present description.

### EXAMPLE 1

160 parts (1.4 mol) of ethyl cyanoacetate, 170 parts of toluene, 20 parts of paraformaldehyde and 0.5 parts of piperidine were mixed and heated in a 0.5 litre, stirred reaction flask, fitted with a thermometer, condenser and a Dean-Stark trap. The mixture was refluxed while stirring until removing the theoretical amount of condensation water. Thereafter, the reaction vessel containing toluene solution of poly(ethyl-α-cyanoacrylate) was cooled down to room temperature. 4 parts of fresh phosphorus pentoxide and 1.5 parts of hydroquinone (weight ratio of 2.67:1.00), as well as 1 part of para-toluenesulfonic acid and 10 parts of ortho-phosphoric acid (weight ratio of 1:10) were then added with good stirring. The toluene solvent was removed under reduced pressure and the residue depolymerised by heating up to 180° C. under a vacuum of 15 Torr. The distillate (102 parts) was collected in a cold flask containing 0.1 part sulphuric acid and 0.05 part of hydroquinone and subjected to additional vacuum distillation to get 94 parts of high-purity (+99%, GC) ethyl-α-cyanoacrylate.

### EXAMPLE 2

Example 1 is repeated except that 120 parts of butyl cyanoacetate were mixed with 23 parts of paraformaldehyde, 0.5 parts of piperidine in 150 parts of toluene using the same reaction vessel and conditions. Depolymerisation at 190° C. under a vacuum of 10 Torr was performed in the presence of 3 parts of P₂O₅, 2 parts of hydroquinone (weight ratio 1.5:1.0), 1 part of para-toluenesulfonic acid and 10 parts of ortho-phosphoric acid (weight ratio 1:10). After purification by additional vacuum distillation, 65 parts of high-purity (+99%, GC) and stabilized butyl-α-cyanoacrylate were obtained.

### EXAMPLE 3

Example 1 is repeated except that 180 parts of 2-ethoxyethyl cyanoacetate were mixed with 32 parts of paraformaldehyde, 0.8 parts of piperidine in 200 parts of toluene using the same reaction vessel and conditions. The resulting poly(ethoxyethyl-α-cyanoacrylate) was depolymerised at 210° C. under a vacuum of 5.0 Torr in the presence of 6 parts of P₂O₅, 3 parts of hydroquinone (weight ratio of 2:1), 0.5 parts of para-toluenesulfonic acid and 5 parts of ortho-phosphoric acid (weight ratio of 1:10). After purification by additional vacuum distillation, 62 parts of high-purity (+98%, GC) and stabilized 2-ethoxyethyl-α-cyanoacrylate were obtained.

The foregoing examples of the preferred embodiments of this invention are presented for purposes of illustration and description. Various modifications or variations are possible. The embodiments were chosen and described to provide the best illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

### REFERENCES CITED

Alan E. Ardis, to Goodrich B. F. Co., NY: ‘Preparation of monomeric alkyl-α-cyanoacrylates’, U.S. Pat. No. 2,467,927 (Apr. 19, 1949).
Frederick B. Joyner and Gary F. Hawkins, to Eastman Kodak Co, NY:
‘Method of making of α-cyanoacrylates’, U.S. Pat. No. 2,721,858 (Oct. 25, 1955).
Frederick B. Joyner and Newton H. Shearer Jun., to Eastman Kodak Co., NY: ‘Preparation of monomeric α-cyanoacrylates’, U.S. Pat. No. 2,756,251 (Jul. 24, 1956).
Robert Rabinowitz, to American Cyanamid Co, Maine: ‘Preparation of alkyl- and aryl-α-cyanoacrylates’, U.S. Pat. No. 3,444,233 (May 13, 1969).
Tien-Lu Wang, Tso-Chi Chiu, Kun-Chuo Chen, to Industrial Technology Research Institute, Chutung, Taiwan: ‘Method for making of alkyl-α-cyanoacrylates from polyalkyl-α-cyanoacrylates’, U.S. Pat. No. 5,436,363, (Jul. 25, 1995).
Neil Hunter Ray and Peter Doran, to ICI Ltd., UK: ‘Preparation of α-cyanoacrylic esters’, U.S. Pat. No. 3,463,804 (Aug. 26, 1969).
Carl J. Buck, to Johnson & Johnson, NY, ‘Modified cyanoacrylate monomers and methods for preparation’, U.S. Pat. No. 4,012,402 (Mar. 15, 1977).
Y. B. Vojtekunas, A. M. Polyakova, K. A. Mager, Y. B. Kohanov, A. I. Vojtkov, to Institute of Organometallic compounds, USSR Academy of Sciences, USSR, ‘Method of making esters of the α-cyanoacrylic acid’, USSR authorship certification 726,086 (March 1980) (In Russian).
Kaspar Schülter and Klaus Marten, to Henkel KGaA, BRD, ‘α-Cyanacrylsäure’, Offenlegungsschrift DE 3415181 A1 (Oct. 31, 1985) (in German).
Alan E. Ardis, to Goodrich B. F. Co., NY: ‘Preparation of monomeric alkyl-α-cyanoacrylates’, U.S. Pat. No. 2,467,926 (Apr. 19, 1949).
John B. Long, to Wingfoot Co., Delaware, ‘Derivatives of fatty acids and method of preparing same’, U.S. Pat. No. 2,391,251 (Dec. 18, 1945).

1. The process for the preparation of alkyl or alkoxyalkyl-α-cyanoacrylates of general formula (1) 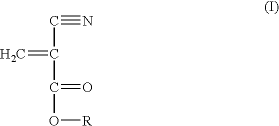
wherein R is a C—C-alkyl or alkoxyalkyl residue
characterized by comprising the depolymerisation of the corresponding poly(alkyl-α-cyanoacrylates) or poly (alkyl-α-cyanoacrylate) in the presence of a multi-component stabilizing system, which comprises
(a) phosphorus pentoxide;
(b) hydroquinone;
(c) ortho-phosphoric acid; and
(d) para-toluenesulfonic acid.

2. A process according to claim 1, characterized by the relation between the phosphorous pentoxide and the hydroquinone being in the range, wt. parts, from 1:10 to 10:1, preferably between 1:1 and 1:5, more preferably between 1:1 and 1:3.

3. A process according to claim 1, characterized by the relation between the ortho-phosphoric acid and the para-toluenesulfonic acid being in the range from, wt. parts, 20:1 and 1:20, preferably between 15:1 and 10:1.

4. A process according to claim 1, characterized by said alkyl-α-cyanoacrylate being ethyl-α-cyanoacrylate and said poly(alkyl-α-cyanoacrylate) being poly(ethyl-α-cyanoacrylate).

5. A process according to claim 1, characterized by said alkyl-α-cyanoacrylate being butyl-α-cyanoacrylate, and said poly(butyl-α-cyanoacrylate) being poly(ethyl-α-cyanoacrylate).

6. A process according to claims 1, characterized by said alkoxyalkyl-α-cyanoacrylate being 2-ethoxyethyl-α-cyanoacrylate, and said poly(alkoxyalkyl-α-cyanoacrylate) being poly(2-ethoxyethyl-α-cyanoacrylate).

7. A process according to claim 1, characterized by the depolymerisation step being performed in the range of temperatures between 100 and 300° C., preferably between 150 and 250° C. and more preferably between 180 and 200° C.

8. A process according to claim 1, characterized by the depolymerisation step being carried out in vacuum in the range from 50 to 0.5 Torr, preferably between 20 and 1 Torr and more preferably between 10 and 5 Torr.

9. The process according to claim 1 characterized by further comprising an additional step of vacuum distillation.

10. Use of the process, according to claim 1, characterized for being applied to the preparation of alkyl or alkoxyalkyl-α-cyanoacrylates of general formula (1).

11. Use of the process, according to claim 10, characterized for being applied to the preparation of technical or medical adhesives based on the alkyl or alkoxyalkyl-α-cyanoacrylates of general formula (1).
